(19) United States
(12) Patent Application Publication
(10) Pub. No.: US 2009/0135372 A1
(43) Pub. Date: May 28, 2009

(54) Modular ocular measurement system

(76) Inventor: Edwin J. Sarver, Carbondale, IL (US)

Correspondence Address: MCHALE & SLAVIN, P.A., 2855 PGA BLVD, PALM BEACH GARDENS, FL, 33410, US

(21) Appl. No.: 12/188,517

(22) Filed: Aug. 8, 2008

Related U.S. Application Data
(60) Provisional application No. 60/954,947, filed on Aug. 9, 2007.

(51) Int. Cl. A61B 3/107

(52) U.S. Cl. 351/212

(57) ABSTRACT

A modular ocular measurement system combines reflection corneal topography with dynamic pupil, limbus, and contact lens measurement, projection corneal-scleral topography, and ocular wavefront measurement to meet the general needs of routine clinical practice, thereby increasing the general commercial viability, as well as the unmet needs of correcting the highly aberrated eye, and in particular the design of wavefront-guided corrections (e.g., soft lenses for the highly aberrated eye, refractive surgery, IOLs, inlays, onlays, etc.).

### RELATED APPLICATIONS

This application claims the benefit under 35 U.S.C. 119(e) of U.S. Provisional Patent Application No. 60/954,947, filed Aug. 9, 2007, entitled MODULAR OCULAR MEASUREMENT SYSTEM, the entirety of which is incorporated herein by reference.

### FIELD OF THE INVENTION

This invention relates generally to apparatus for use in determining the front shape and power of the cornea of a human eye and thus facilitating the diagnosis and evaluation of corneal anomalies, design and fitting of contact lens, and the performance of surgical procedures. The present invention also relates to the field of measurement of the refractive characteristics of an optical system, and more particularly, to automatic measurement of the refractive characteristics of the human or other animal eye and to corrections to the vision thereof.

### BACKGROUND OF THE INVENTION

Most state-of-the-art clinical instruments used to design wave front-guided corrections are typically either devoted to wave front sensing (e.g., Wave front Sciences COAS), or corneal topography (e.g., Optikon Keratron) and can be tied specifically to a laser platform (e.g., AMO/VISX WaveScan). Repeated requests from the community for instruments to link wave front sensing with corneal topography has resulted in combined instruments (i.e., the Nidek OPD-Scan and the Topcon KR9000).

These devices are well suited for measuring the normal eye, have variable results on the abnormal eye, and have limited features for designing a variety of wave front-guided corrections (e.g., wave front-guided soft contact lenses, onlays, inlays, IOLs).

Consider the correction of eyes with large amounts of high order wave front error (HO WFE) (e.g., keratoconus, pellucid marginal degeneration, penetrating keratoplasty, poor refractive surgery outcomes, etc.). These eyes have decreased visual performance which is increasingly aggravated with pupil dilation. The current gold standard correction for these patients is a rigid gas permeable (RGP) contact lens. A RGP lens reduces the HO WFE by providing a new first refracting surface and filling of the space between the RGP lens and cornea with tears, which are closely index matched to both the lens and cornea. Two major factors reduce the utility of RGP lenses at correcting HO WFE. First, the index matching is not perfect. Second, HO WFE originating from the corneal back surface cannot be reduced by tear index matching. As a result, RGP correction improves vision but does not typically improve visual performance to normal levels. Additionally, RGP lenses fall far short in meeting the additional patient needs of wear time and comfort, decreasing the patient's quality of life. Illustrating this point, Crews and Driebe note that decreased RGP lens wear time and lens discomfort are major causes for corneal transplant surgery in patients with keratoconus.

Soft contact lenses increase comfort and wear time dramatically. Recent advances in contact lens research have demonstrated the capability to manufacture state of the art custom wavefront-guided soft contact lenses (WGSLs) for the treatment of highly aberrated eyes. These lenses, in the handful of patients tested to date, provide equal or better acuity compared to habitual RGP corrections. Unfortunately, the capability to effectively and efficiently design custom WGSL corrections in the clinical environment is severely limited by current instrumentation. For example, current instrumentation does not reliably report Shack/Hartmann ocular wavefront data on the highly aberrated eye due to spot dropout.

A similar situation exists for corneal elevation or dioptric topography measurements made with Placido based technology. Highly aberrated eyes distort the rings of current Placido instruments, making edge tracking difficult leading to data drop out or data errors. This fact is well illustrated in test-retest repeatability on keratoconic eyes for three clinically available instruments (EyeSys Model II, Dicon CT 200 and the Keratron Corneal Analyzer) and was shown to be very poor. Further, registering this noisy data to an independent coordinate system from a wavefront error measurement is uncertain at best.

Placido corneal topography data does not cover the entire cornea and the area covered decreases as corneal curvature increases. When designing a WGSL it is useful to contour the back surface to conform to the cornea and onto the sclera. Such a design increases stabilization of the lens on the eye.

The benefit of a wavefront-guided correction decreases as registration errors between the wavefront-guided correction and the wavefront error increase. For example, contact lenses translate and rotate on the eye. Depending on the particular aberrations involved, the magnitude of each type of aberration and the amount of lens movement, a wavefront correction can be designed to provide optimal average retinal image quality. Data detailing the movement of a soft lens on an individual's eye allows the lens designer to first design optimal stabilization strategies and second, given the residual movement, design an optimal correction for that patient.

The benefit of a wavefront-guided correction also decreases if the pupil diameter naturally dilates to a diameter larger than the correction. Physiological pupil diameters vary widely between individuals for any given luminance level and as a function of age. Additionally, the location of the pupil center with respect to the optics of the eye varies slightly as the pupil varies its diameter. To optimally design a wavefront-guided correction, regardless of type, the designer needs to know how pupil size and location vary.

The MOMS proposes to overcome these instrumentation problems and limitations by combining the following features into a single instrument. MOMS combines reflection corneal topography (with dynamic pupil, limbus, and contact lens measurement), projection corneal-scleral topography, and ocular wavefront measurement.

### SUMMARY OF THE INVENTION

An objective of the present invention is to provide an ocular measurement system for determining the front shape and power of the cornea of an eye by employing a placidocorneal topography measurement system, a projection corneal topography measurement system and an ocular wavefront measurement system.

Another objective of the invention is employ dynamic pupil, limbus, and contact lens measurements.

Still another objective of the invention is to teach the use of measurements taken along a common coordinate system.

Another objective of the invention is to teach the use of reflection corneal topography with multi-resolution sinusoidal profile pattern.

Another objective of the invention is to teach the use of projection topography using Scheimpflug geometry for improved depth of field.

Another objective of the invention is to teach the use of variable resolution ocular aberrations using selectable Hartmann screens.

Another objective of the invention is to teach the use of scanning ocular aberrations using spinning, tilted parallel plate.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a schematic diagram of the optical system of the present invention;

FIG. 2 is a Placido target geometry;

FIG. 3 is a profile of a symbolic ring to be paced on the Placido target;

FIG. 4 is a curve of the combination of frequencies employed in a multi-resolution Placido target;

FIG. 5 is the camera system employed for the Placido corneal topographer;

FIG. 6 illustrates the feature points for a given semi-meridian;

FIG. 7 illustrates a sample intensity profile for a 8.0 mm sphere;

FIG. 8 illustrates a test for extremal points local maximum and local minimum;

FIG. 9 illustrates a typical spherical or conic case where extrema line up with low resolution peaks;

FIG. 10 illustrates a double imaging of single refection target point P;

FIG. 11 illustrates a basic setup for arc-step iteration;

FIG. 12 is a sinusoidal amplitude grating at the image plane at the cornea of an eye;

FIG. 13 illustrates the basic geometry for the projection corneal topography system;

FIG. 14 illustrates the calculation of x-location of the surface point s for the projection corneal topography system;

FIG. 15 is the Fourier transform of the warped sinusoid with three primary peaks;

FIG. 16 is the cutoff frequency for the low-pass filter;

FIG. 17 illustrates a side view of a ray tracing of a tilted parallel plate;

FIG. 18 illustrates a micro lens array and a Hartmann screen;

FIG. 19 illustrates the propagation of a plane wave from an aperture;

FIG. 20 illustrates a sample image from a focal plane of a Hartmann screen; and

FIG. 21 illustrates an unfolded ZEMAX paraxial ray tracing of the relay lens.

### DETAILED DESCRIPTION OF THE INVENTION

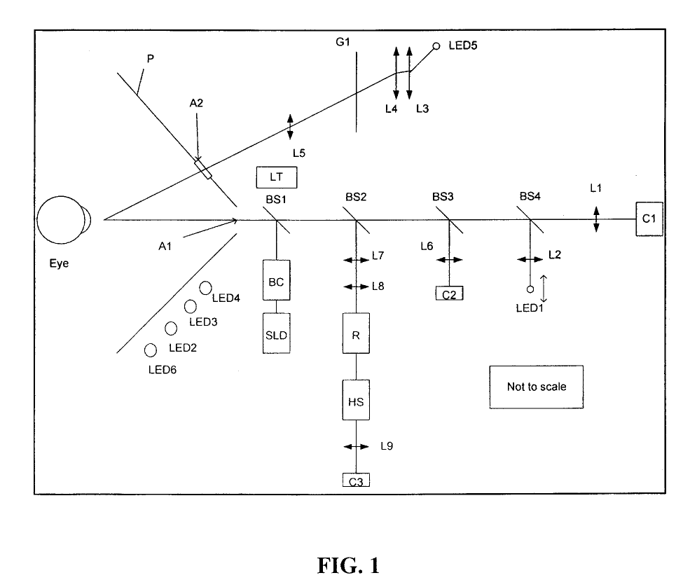

The basic optical system layout is shown in FIG. 1. Each of the items labeled in FIG. 1 are listed and described in Table 1.

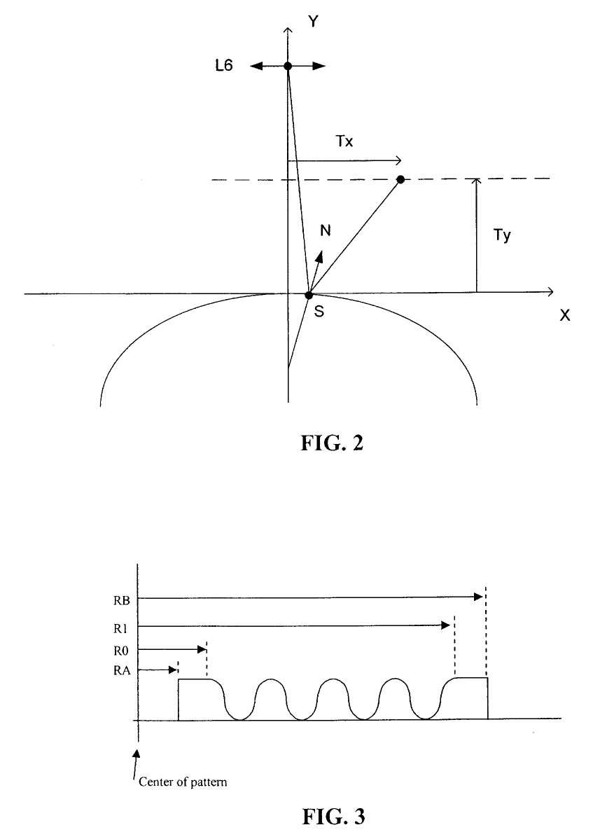

TABLE 1
Listing and description of all items identified
in the basic optical system in FIG. 2.

| Item | Description |
|---|---|
| A1 | Central aperture in Placido |
| A2 | Aperture in Placido for projected sinusoidal grating |
| BC | Beam conditioning for SLD |
| BS1 | Pellicle beam splitter for SLD |
| BS2 | Hot mirror (reflects 830 nm) for wavefront sensor |
| BS3 | Green reflector for projection topography sensing |
| BS4 | Reflector for fixation source |
| C1 | Camera for sensing Placido topography, contact lens, |
| | and pupil measurement |
| C2 | Camera for sensing projection topography |
| C3 | Camera for sensing wavefront |
| Eye | Patient's eye to be measured |
| G1 | Sinusoidal grating for projection topography |
| HS | Hartmann screens and selection mechanism |
| L1 | Lens to focus reflected target, contact lens and pupil |
| | onto camera C1 |
| L2 | Lens for focusing fixation LED |
| L3, L4 | Relay lens for sinusoidal grating illumination |
| L5 | Projection lens for sinusoidal grating |
| L6 | Lens to focus projected grating onto camera C2 |
| L7, L8 | Relay lens to place Hartmann screen conjugate to the |
| | eye's wavefront |
| L9 | Lens to focus the Hartmann screen focal plane onto |
| | camera C3 |
| LED1 | Fixation source (555 nm) |
| LED2 | Placido illumination (650 nm) |
| LED3 | NIR for pupil measurement (780 nm) |
| LED4 | Visible LED to illuminate the pupil during pupil |
| | measurement (TBD nm) |
| LED5 | High bright Cyan LED for projection CT (505 nm) |
| LED6 | Illumination for lens tracking (555 nm) |
| LT | Light trap for SLD |
| P | Placido profile |
| R | Rotator to provide wavefront scanning |
| SLD | Super luminescent diode used as a source for the |
| | wavefront sensor (830 nm) |

The optical system shown in FIG. 1 contains three primary modules: a) Placido corneal topography including dynamic limbal detection, pupil detection and contact lens detection; b) projection corneal topography; c) and ocular wavefront. We describe the basic operation of each of these modules in this section and provide design details in the following sections.

To begin an exam, the patient's eye is positioned in the location of the Eye in the optical system diagram. The fixation led LED1 is located at the focal point of lens L2. When LED1 is illuminated, rays from it will propagate through L2 parallel to the optical axis and will appear to the patient to be at optical infinity. This is the single fixation source used for the acquisition of all data (corneal power, corneal elevation, ocular aberrations, etc.).

Placido Corneal Topography. To acquire a Placido corneal topography exam, first a set of LEDs (identified as LED2 in the system diagram) is turned on to back illuminate the multi-resolution concentric ring Placido P. Light from the Placido target is specularly reflected off the cornea and some of the rays will enter the aperture A1, pass through beam splitters BS1-BS4 and be refracted by lens L1. These rays are focused by lens L1 onto the sensor of camera C1. The object plane for camera C1 is about 4 mm behind the corneal vertex as the radius of curvature of the cornea is approximately 8 mm. From the captured image we can compute corneal power data as well as detect the limbus contour and locate its center. The pupil center and diameter can also be measured from an image captured from camera C1 as well as pupil center location with respect to the limbal center. In this case, the pupil is illuminated with LED3 through the Placido to enhance the contrast of the pupil relative to the iris. Also, the pupil response as a function of luminance can be controlled using visible stimulus LED4. In a similar manner the position and angular orientation of a contact lens (with specific fiducial marks) can be measured from images captured by C1. In this case, the contact lens and fiducial marks are illuminated by LED6.

Projection Corneal Topography. Prior to the acquisition of a projection corneal topography exam, it is necessary to instill fluorescein into the patient's eye. To acquire a projection corneal topography exam, the projection source LED5 is illuminated. Light from LED5 passes through the illumination relay lenses L3 and L4 and then passes through the sinusoidal amplitude grating G1. The illuminated grating G1 is then brought into focus at a location approximately 2 mm behind the corneal vertex by projection lens L5. Rays from lens L5 pass through the side aperture A2 of the Placido target P. Due to the orientation of the lens L5 and grating G1, the focal plane at the eye is in the desirable state of being approximately perpendicular to the optical axis of the instrument. This special setup of the projection system is called the Scheimpflug condition. As the light is projected onto the patient's eye, it will cause a diffuse fluorescent reflection due to the fluorescein placed in the patient's eye prior to the exam. Rays from these diffuse reflections pass through aperture A1 and beam splitters BS1 and BS2. When these rays encounter beam splitter BS3, they are re-directed toward lens L6. Lens L6 focuses these rays onto camera sensor C2. From the captured image we can compute the elevation of the cornea and a portion of the sclera.

Ocular wavefront. To begin an ocular wavefront exam, the super luminescent diode SLD is illuminated. Rays from the SLD are passed through the beam conditioner BC which contains a small aperture to limit the size of the collimated beam. A small portion (8%) of the collimated beam is reflected off pellicle beam splitter BS1 and directed toward the eye. The remainder of the collimated beam passes through BS1 and is trapped by light trap LT. After reflecting off BS1 and being directed toward the eye, the collimated beam passes through the aperture A1 and enters the eye where it forms a diffuse reflection on the retina. Rays from this diffuse reflection propagate back out of the eye and back through aperture A1 and pass through beam splitter BS1. These rays then reflect off beam splitter BS2 where they are directed toward relay lenses L7 and L8. The lenses L7 and L8 relay the wavefront from the plane of the eye's entrance pupil to the selectable Hartmann screen wavefront sensor HS. Prior to reaching HS, the wavefront passes through the beam rotator R where the wavefront can be slightly repositioned orthogonal to the local optical axis. This repositioning permits higher spatial sampling of the wavefront from sequential captures of HS images. The spots from the HS are warped by the relayed and repositioned wavefront to the focal plane of the HS. From there, lens L9 focuses the warped spots onto the sensor of camera C3. From the captured images of the HS focal plane we can compute the ocular wavefront aberrations of the patient's eye.

Now that we have described the basic operation of the combined system, we turn our attention to design details of the individual functions of the breadboard. The three modules to be discussed are the Placido corneal topography with limbal, pupil and contact lens detection, the projected corneal topography, and the ocular wavefront modules. The Placido corneal topography module is responsible for collecting power measurements of the cornea as well as providing static and dynamic measurements of the limbus contour, pupil and contact lens. The main topics to be discussed are the Placido target, the camera optics, and some computational aspects.

The Placido target refers to the pattern that is back illuminated and reflected off the cornea. The first two parameters (endpoints of the Placido target profile) to be computed concern the physical size of the target. The basic geometry for these calculations is illustrated in FIG. 2. In this figure, a target (Placido) point at (Tx, Ty) is reflected off the cornea at surface point (Sx, Sy). The reflected ray then passes through lens L6.

In FIG. 2, the distance to L6 is about 300 mm, thus the ray between the center of lens L6 and the surface point S is essentially parallel with the instrument axis (y-axis). Using this approximation (only for the sizing of the Placido—approximation is not used in the reconstruction algorithm), the x location of the target point Tx can be found from equation (1).

$$Tx = Sx + Ty \cdot tan [ 2 {sin}^{- 1} ( \frac{Sx}{R} ) ] \qquad ( 1 )$$

In this equation, R is the radius of the reference sphere. For our calculations, R=8 mm. The calculations at the end points of the target profile are:

R=8 mm, Ty=96 mm, Sx=0.45 mm→Tx=11.75 mm

R=8 mm, Ty=25 mm, Sx=4.75 mm→Tx=85.75 mm

Thus, the profile of the target face has endpoints: (11.75, 96) and (85.75, 25). When this profile is rotated about the optical axis it creates a three-dimensional cone shape typical of commercial corneal topographer systems. This geometry will ensure that when we measure an 8 mm sphere the inner ring will correspond to a measurement zone of 0.9 mm on the sphere and the last ring will correspond to a measurement zone of 9.5 mm. These measurement values are consistent with the testing described below in the demonstration of feasibility.

A symbolic ring profile to be placed on our Placido is illustrated in FIG. 3. Note that this profile is with respect to the reference surface, not the reflection target. The resulting captured reflection target image will be a warped version of this profile. The starting and stopping edges at RA and RB were added to make a clear starting and stopping point for the pattern.

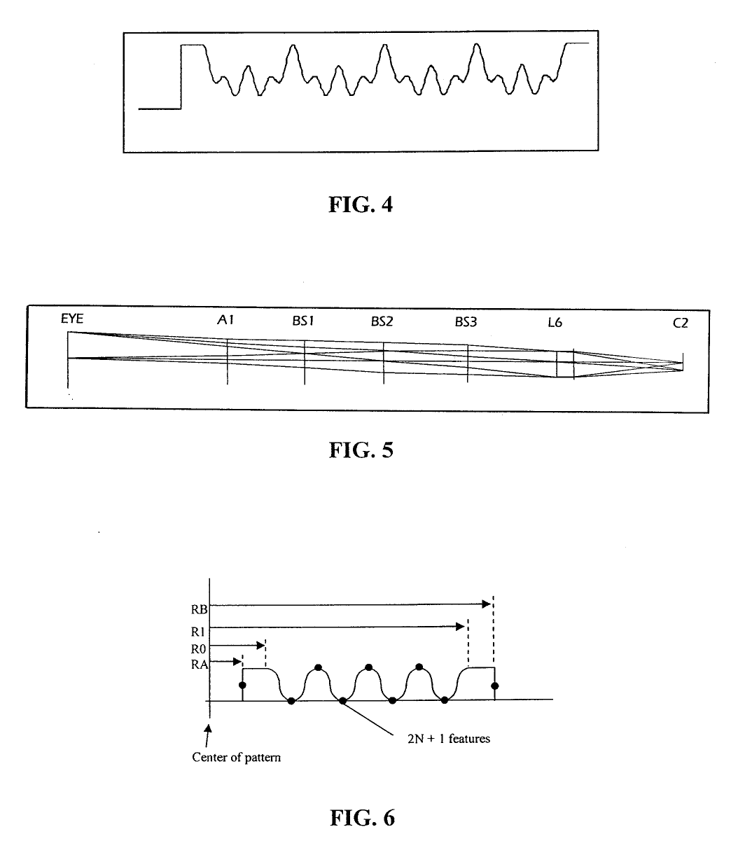

For a multi-resolution target, we combine three frequencies, with each subsequent frequency twice the previous frequency. The resulting amplitude profile illustrated in FIG. 4.

This profile provides what can be described as low-, medium-, and high-resolution rings, hence the description of the results system as multi-resolution corneal topography. To determine how the pattern is warped onto the Placido face, we use the same geometry illustrated in FIG. 2. Here, we scan points (pixels) along the reflection target and convert the location to mm. For one of these points P, we find the location at the reference surface (the am sphere) that reflects the target point to the center of the lens L. Given the reflection point S, we look up the intensity profile (from the curve in FIG. 5) based upon the x-value of the surface location. This mapping shows how to apply the pattern to the Placido face. The camera selected is a 1394b (Firewire) ⅓ inch (4.8×3.6 mm) monochrome camera. The camera system for the Placido corneal topographer is illustrated in FIG. 5. The desired horizontal field of view (for all uses of this camera) at the eye is 15 mm. We extend this to 17 mm so we have plenty of space on the sides of the image. This leads to a vertical field of view of 12.75 mm (17×0.75) and a magnification of:

$$m = \frac{4.8}{17} = 0.2824 \qquad ( 2 )$$

The lens L1 in FIG. 2 is actually two achromatic lenses selected to reduce distortion compared to a single lens. The first lens is a 300 mm focal length object lens. This focal length is required due to the long distance from the eye to the lens L1. Given the desired magnification, we can compute the focal length of the second (image) lens as

imageLensF=m×300=84.7≈85 (3)

The length of the diagonal at the object plane is 21.25 mm. This diameter is used in the ray trace analysis to calculate element size to prevent vignetting. Ray tracing this field height in ZEMAX yields the image shown in FIG. 6. Using this ray tracing, the diameter of the elements required to prevent vignetting was found to be as listed in Table 2.

TABLE 2

| Element | Diameter |
|---|---|
| A1 | 17.5 |
| BS1 | 15.6 |
| BS2 | 13.8 |
| BS3 | 12 |
| L6 - 300 fl | 10 |
| L6 - 85 fl | 10.6 |
| Image plane | 6.02 |

The basic steps taken to process the acquired Placido corneal topography image will now be described. The processing proceeds in a sequential fashion using the following steps: 1) Center detection; 2) Sub-pixel center estimation; 3) Feature detection (edges and peaks); 4) Sub-pixel feature estimation; and 5) Surface reconstruction.

The rough center region is easily found by looking for a M×M square with the darkest average intensity within the central search region of interest (ROI) (central Width/4×Height/4 region).

Sub-pixel center estimation. Once the rough center is found, several scans (in different angular directions) in the intensity array are made from the center outward. For each of these vector scans, the vector is differentiated using a filter impulse response of (−1, −1, −1, 0, 1, 1, 1). The first large peak is taken as the location of the edge of the center circle. The (x,y) location of each of these circle edge points (one point per angular direction) is saved. A circle is then fit to these points by solving for the coefficients a, b, and c using the system of equations indicated in equation (4).

a(xᵢ²+yᵢ²)+bxᵢ+cyᵢ=1 (4)

The center of this analytical equation of a circle is taken as the center of the ring pattern.

After the center has been located to sub-pixel accuracy, we are ready to find all the ring edges in all radial directions from the center of the image. Feature detection is performed for each one-dimensional semi-meridian extracted from the image. The semi-meridian originates at the center previously found above. The features found are: the center edge, the individual peaks and valleys of the sinusoid and the peripheral edge. These feature points are illustrated by the black dots in FIG. 6.

Note that there are 2N+1 feature points along a semi-meridian where N is the number of cycles of the sinusoid for a given resolution profile. We proceed by first finding the lowest frequency profile features.

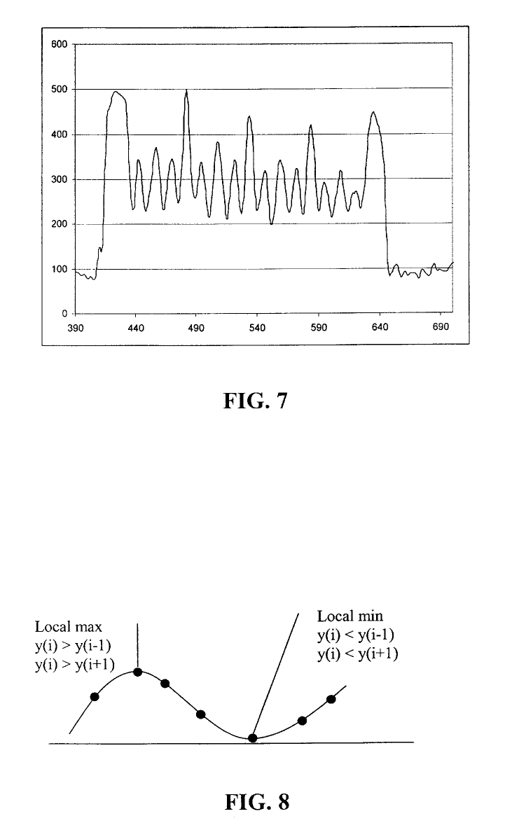

An actual example profile is shown in FIG. 7. The starting and stopping edges are easily found using the same differentiation/magnitude detector that we employed in the center finding routine.

After the starting and stopping edges and low resolution peaks are found, we scan across the vector looking for all extremal points. Extremal points include the peaks and valleys of the sinusoids. The basic algorithm for extremal points is illustrated in FIG. 8. If the current point is greater than its neighbors to the left and right, it is a local maximum. If the current point is less than its neighbors to the left and right, it is a local minimum.

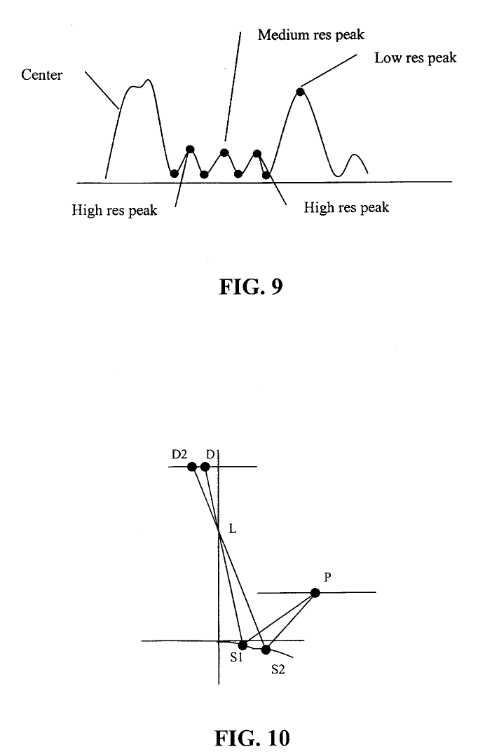

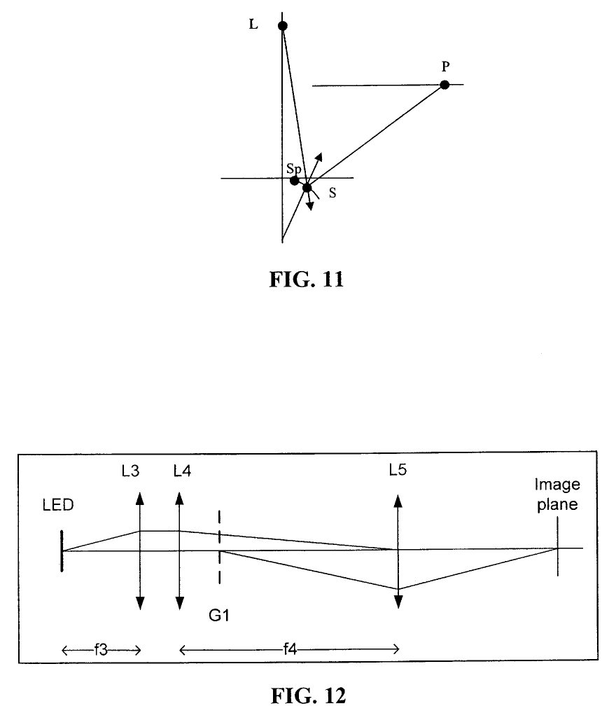

The extrema found along the semi-meridian for the intensity vector will have peaks in common with the low frequency peaks found as described above. We also know they should appear at specific locations. If all goes as planned, we have the case illustrated in FIG. 9.

If we have the case where the expected locations in the extrema vector line up with the locations of the low resolution peaks, all is well and we may proceed to the sub-pixel estimation processing. However for the case of a bi-sphere where the central region has smaller radius of curvature than the periphery, a serious problem can arise. In the case illustrated in FIG. 10, a single point from the reflection target is reflected twice from the bi-sphere surface: One reflection from the steep central region and another from the flatter peripheral region. This is not due to an instantaneous change in curvature, but just one region with much steeper curvature than the other. A real-world example of this may arise from a particular presbyopic or hyperopic LASIK corrected cornea. Once detected, we use the highest resolution that does not exhibit the double reflection problem and continue with reconstruction.

Surface reconstruction. The midpoint arc-step reconstruction is used to reconstruct the surface. It is performed much like a standard arc-step algorithm, with the exception that following an arc-step iteration a surface midpoint step is performed. Our arc-step iteration is illustrated in FIG. 1. In this figure, we are given a previous point Sp (at the first ring this corresponds to the vertex at 0,0), the lens location L, the reflection target point P, and the reflection vector L-S. Our goal is to vary the radius of the circle so that the incident and reflection angles are equal. This optimization is performed using Newton iteration and usually converges in 3 to 4 iterations. At each step we save the location of the point and the axial radius of the arc. Following the arc-step iteration, we find the starting point Sp for the next iteration, etc.

It is simply expressed as in equations (5) for the center ring and for subsequent rings. Here c is the center of curvature ra is the axial radius, z is the surface sag point at S and x is the radial distance from the center to S.

Starting ring=0:

c₀=−ra₀

z₀=c₀+\sqrt{ra₀²−x₀²} (5)

By examination of equation (6) it is apparent that the algorithm behaves as simultaneously computing the average of two arc-step algorithms for rings 1 to N−1.

$$Rings n = 1 , … , N \text{-} 1 \text{:} \\ \begin{matrix}\begin{matrix}\begin{matrix}{c}_{n} = {z}_{n - 1} - \sqrt{{ra}_{n}^{2} - {x}_{n - 1}^{2}} \\ {z}_{n 0} = {c}_{n} + \sqrt{{ra}_{n}^{2} - {x}_{n}^{2}}\end{matrix} \\ {z}_{n 1} = {c}_{n - 1} + \sqrt{{ra}_{n - 1}^{2} - {x}_{n}^{2}}\end{matrix} \\ {z}_{n} = \frac{{z}_{n 0} + {z}_{n 1}}{2}\end{matrix} \qquad ( 6 )$$

The projection corneal topography module is responsible for measuring the elevation data of the cornea as well as the limbus and a small portion of the sclera. Our method is similar to the Fourier profilometry method of Takeda and Mutoh.

Illumination and projection optics. The illumination for the sinusoidal amplitude grating is based on a two-element relay lens and an LED with its lens removed. This is illustrated in the left side of FIG. 12. The LED is a high brightness (30 lumens), 505 nm LED. Light rays from the LED are transmitted by lens L3 (a 30 mm condenser lens). Parallel rays are then transmitted through lens L4 and are brought into focus in the clear aperture of lens L5. The sinusoidal amplitude transmission grating G1 is located about 10 mm in front of lens L4 where it is evenly illuminated by the light coming from L4. Projection lens L5 then focuses the image of G1 onto the image plane 2 mm behind the corneal vertex. This setup makes a very non-uniform illumination source appear very uniform at the image plane and efficiently transmits the light that enters L3 to the clear aperture of L5. In the figure we show these elements centered on the optical axis. In reality, these elements are offset so that the grating G1 is projected to the image plane so that it is perpendicular to the instrument axis.

Sinusoidal amplitude grating. The sinusoidal amplitude grating has a frequency of 0.2 c/mm at the image plane at the cornea. The grating is very similar to a Ronchi ruling (parallel opaque bars with 50% duty cycle), except the profile of the grating is sinusoidal instead of a square wave.

The camera optics are the same as described under Placido Corneal Topography.

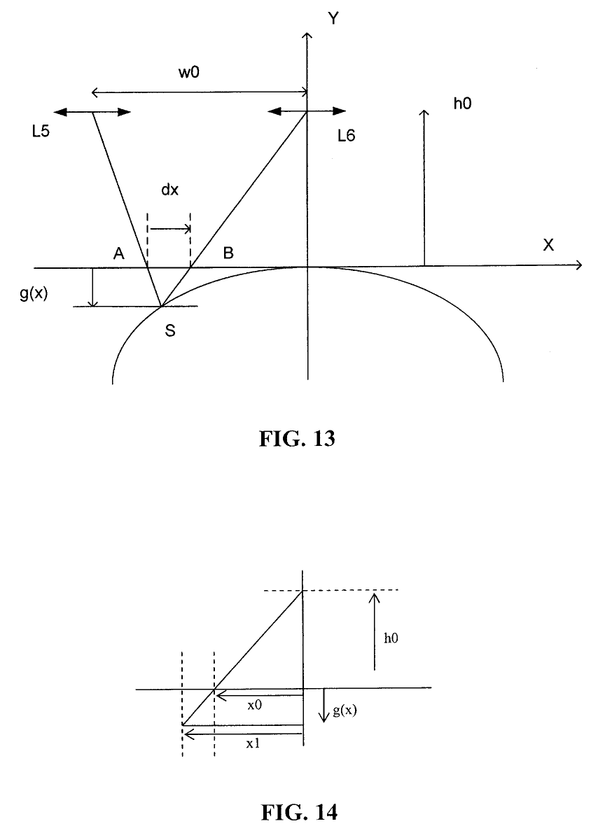

The basic geometry for the projection corneal topography system is illustrated in FIG. 13.

In this figure, the projection lens L5 is located a distance w0 from the instrument optical axis (y-axis). Both the projection lens L5 and the camera lens L6 are at the same distance h0 from the plane tangent to the corneal vertex. A ray from the projector intersects the x-axis at point A and the cornea at point S where it makes a diffuse reflection due to the fluorescein in the tears. The camera observes this diffuse reflection at S according to the ray from S to the camera lens L6. The point B is the intersection of the camera observation ray with the x-axis. The height of the surface is given by the function g(x). As drawn in FIG. 13, g(x) has a negative value. Using similar triangles (L5 S L6) and (A S B), we can show the relationship between the height g(x) at S and the distance between A and B is

$$d x = \frac{g ( x ) \times w 0}{g ( x ) - h 0} \qquad ( 7 )$$

In the figure above, a profile of the sinusoidal grating is projected along the x-axis. As viewed by the camera through lens L6, the sinusoidal grating is warped according to the distance dx. When the height profile g(x) is a flat surface in the plane of the x-axis, the distance between points A and B is zero, and a perfect sinusoidal pattern is observed. If we let w(x) represent the local distance dx, then the warped profile can be expressed

$$\begin{matrix}\begin{matrix}c ( x ) = \frac{1}{2} + \frac{1}{2} cos ( \frac{2 π}{p} ( x + w ( x ) ) ) \\ = \frac{1}{2} + \frac{1}{2} cos ( \frac{2 π}{p} x + phi ( x ) )\end{matrix} & ( 8 ) \\ phi ( x ) = \frac{2 π}{p} w ( x ) & \end{matrix}$$

If we can recover the phase function phi(x), then the surface elevation g(x) can be found from

$$g ( x ) = \frac{h 0 phi ( x )}{phi ( x ) - \frac{2 π}{p} w 0} \qquad ( 9 )$$

When the camera ray is detected, we know the x-location of point B, but we want the x-location of the surface point S. This is calculated as follows:

Using similar triangles we have

$$\frac{x 0}{h 0} = \frac{x 1}{h 0 - g ( x )} \\ x 1 = \frac{x 0}{h 0} ( h 0 - g ( x ) ) \qquad ( 10 )$$

To recover the phase function phi(x) from the rows of the captured image, we first note that the Fourier transform of the warped sinusoid has the form indicated in the FIG. 16 below.

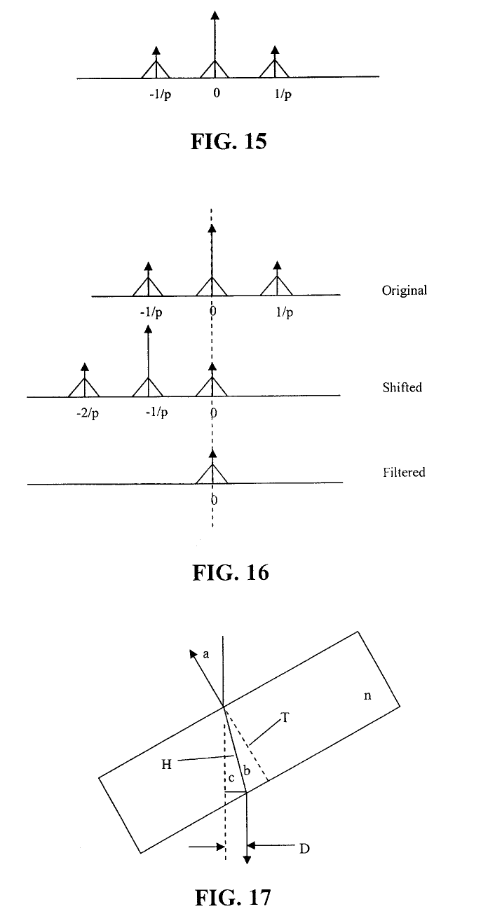

In FIG. 15 there are three primary peaks. Two of the peaks correspond to the cosine function. The spreading of frequencies about the primary peaks is due to the phase warping function that we wish to recover. Our strategy is to shift the spectrum so that the neighborhood at 1/p is centered at the origin and then low-pass filter the remainder so that only the single neighborhood remains.

The shift operation is performed by multiplying the captured warped sinusoidal profile by a complex exponential as in

$$f ( x ) = {c ( x )}^{*} exp ( j \frac{2 π}{p} x ) \qquad ( 11 )$$

The subsequent filter operation is performed using FFTs as follows:

Take the FFT of f(x)

Zero out of band samples

Take the inverse FFT

The cutoff frequency for the low-pass filter shown in FIG. 16 is 1/(2p). The wrapped phase function wrapped_phi(x) is the phase of this shifted and low-pass filtered signal. To recover the unwrapped phase function we perform the unwrapping algorithm below starting in the center and processing first to the right and then returning to the center and processing to the left. Simple unwrapping algorithm steps are as follows:

wphi(n) = wrapped phase
N = length of discrete array to process
phi = desired unwrapped phase
1. phi(N/2) = wphi(N/2)
2. for n=N/2+1 to N−1 do the following

a. del = wphi(n) − wphi(n−1)
b. if | del | < 0.9 × 2 × π then set phi(n) = phi(n−1) + del
c. else if del < −0.9 × 2 × π then set phi(n) = phi(n−1) + del + 2 × π
d. else if del > 0.9 × 2 × π then set phi(n) = phi(n−1) + del − 2 × π

Once we have recovered the unwrapped phase phi(x), we apply equations (9) and (10) to extract the height values and the adjusted surface locations (the x-locations of the height values).

The ocular wavefront module is used for measuring the ocular wavefront aberrations of the eye. The primary elements of this module are the light source (SLD and optics), the sensor path relay lens, the sensor path wavefront rotator, the sensor path adjustable Hartmann screen, and the sensor path camera optics.

SLD optics. The current regulated SLD has a wavelength of 830 nm in a TO-56 package. The output of the SLD is conditioned using an off-the-shelf collimation assembly. The SLD is integrated with the collimator, and then the collimator is adjusted using a simple spanner wrench. The final collimator lens position is fixed using LocTight on the positioning threads. A 0.75-mm aperture is placed at the output of the collimation tube to limit the diameter of the beam entering the eye. This small beam size allows us to easily control the specular reflection of the SLD beam at the corneal first surface by slightly moving the instrument in a lateral direction (up, down, left, or right) if the reflection is seen on a patient's eye.

Safe Light Levels. We follow ANSI Z136.1-2000 to compute safe light levels for the measurement beam at the cornea. The NIR (830 nm) measurement ray can be ON for a relatively long time. To be conservative, we will use 3×10⁴ seconds as the duration for the NIR light. This equates to staring continuously at the source for 8.3 hours. The limiting aperture to use in the computations is taken from ANSI Z136.1, Table 8, and is 7.0 mm so that the area (A) is 0.3848 cm². From ANSI Z136.1, Table 5a, the maximum permissible exposure (MPE) for NIR wavelengths (700 to 1050 nm) and exposure duration (10 to 30,000 s) is given by equation (12).

MPE_{N}=C_{A} mWcm−2 (12)

The value for C_{A} for our NIR wavelength is found in Table 6 of ANSI Z136.1 to be

C_{A}(0.83)=10^{2(λ-0.700})=10^{2(0.830-0.700)}=1.82 (13)

The NIR MPE in units of mW is found to be:

MPE_{N}(0.83)=C_{A}(0.83) mWcm⁻²×A cm²=1.82 mWcm⁻²×0.3848 cm²=0.70 mW (14)

Thus, the 830 nm NIR eye illumination source can be viewed for 8.3 hr at 0.700 mW. While the calculated safe limit is rather large at 0.700 mW, we plan to limit the power at the eye to around 100 μW in line with other similar wavefront systems.

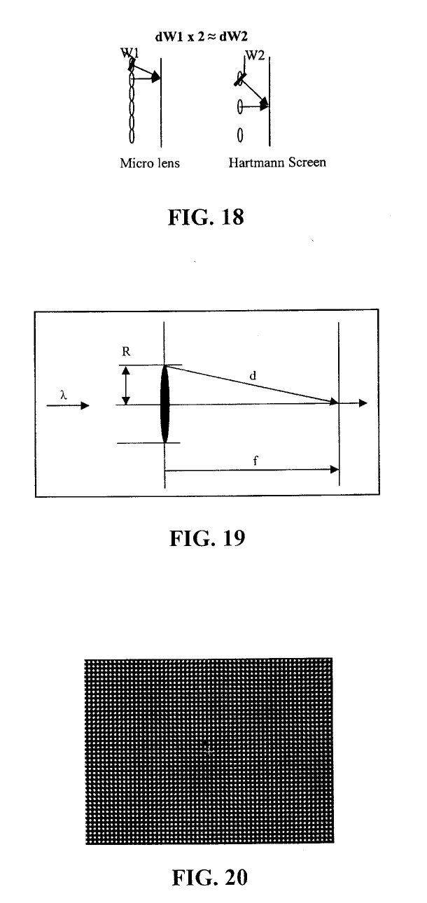

Rotator optics. One means to increase the spatial resolution of a Hartmann screen wavefront sensor is to scan the incoming wavefront over the sensor. This scanning could be a conventional x,y raster scanner, but this requires two scanners that must change direction at some point. Another strategy is to use a rotating parallel plate(s). With one plate, a vast increase in the spatial sampling can be accomplished. The side view of a ray tracing of a tilted parallel plate is shown in FIG. 18.

In FIG. 17, the incoming ray makes an angle “a” with the normal to the parallel plate. The ray is then refracted at the top surface toward the normal inside the glass. At the bottom surface the ray emerges parallel to its original path and has been shifted a distance D. The thickness of the glass plate is T, and the index of refraction of the glass is n. The shifted distance D can be calculated as given in (Mouroulis 1997):

$$D = T sin ( a ) ( 1 - \frac{cos ( a )}{\sqrt{{n}^{2} - {sin}^{2} ( a )}} ) \qquad ( 15 )$$

Our wavefront sensor is a Hartmann screen. The sensor also has a high dynamic range by virtue of missing apertures compared to a standard micro lens array as illustrated in the FIG. 18.

As seen in FIG. 18, if the micro lens array and the Hartmann screen have about the same focal length, the Hartmann sensor will permit about twice the wavefront slope compared to the micro lens array. This is because the micro lens array has tightly packed lenses and the Hartmann screen apertures are separated by opaque regions.

To see how the apertures can act as lenses for a specific wavelength, we consider the effects of constructive interference. A side view of an aperture is illustrated in FIG. 19.

In FIG. 19 a plane wave of wavelength λ propagates from left to right and encounters an aperture of semi-diameter R. An observation plane is located a distance f downstream from the aperture. For constructive interference we want

$$d - f = \frac{λ}{2} \qquad ( 16 )$$

Using the upper right triangle we also have

R²+f²=d² (17)

Combining (21) and (22) and solving for the distance f we have

$$f \approx \frac{{R}^{2}}{λ} \qquad ( 18 )$$

Equation (18) shows the focal distance for the aperture used as a lens. A Hartmann screen with apertures spaced p mm apart has aperture diameter 2R=p/2 mm. For an SLD wavelength of 830 nm the “focal length” of a Hartmann screen with p=0.25 mm is about 4.7 mm. A sample image captured from the focal plane of one of our Hartmann screens is shown in FIG. 20.

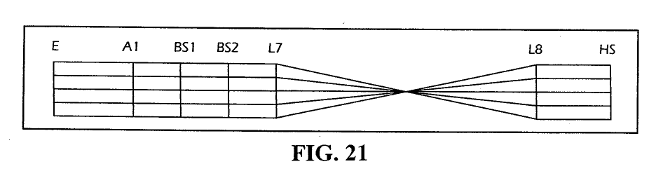

In this figure, we show a captured image for a plane wave input with a 14×10.5 mm field of view. In the MOMS project we will use the same concept with multiple Hartmann screens (each having twice the inter-aperture spacing than the previous—e.g., period=1p, 2p, 4p for three Hartmann screens) to achieve much larger dynamic ranges. Relay lens. The relay lens is responsible for making the wavefront at the entrance pupil of the eye conjugate with the Hartmann screen. An unfolded ZEMAX paraxial ray tracing of the relay lens is illustrated in FIG. 21.

In this unfolded version of the ocular wavefront sensor path, E is the location of the eye's entrance pupil, A1 is the Placido aperture, BS1 and BS2 are beam splitters, L7 and L8 are 175 mm relay lenses, and HS is the Hartmann screen. The distance from E to L7 is 250 mm, the distance from L7 to L8 is 350 mm, and the distance from L8 to HS is 100 mm. This results in a 1:1 relationship between the size of the wavefront at the entrance pupil and the HS.

We can determine the size of the optical elements to prevent vignetting by considering the extremes (5 D of hyperopia, 15 D of myopia) of allowable defocus at the maximum diameter size of 10 mm. Performing this defocused ray tracing, we arrive at the element sizes in Table 4.

TABLE 4
Size of required clear apertures for 10 mm pupils for wavefront
defocus at extremes of desired measurement range.

| | | | Required Optic |
|---|---|---|---|
| Element | 10 D hyperope | 15 D myope | Diameter |
| E | 10 | 10 | 10 |
| A1 | 20 | 5 | 20 |
| BS1 | 25 | 5 | 25 |
| BS2 | 30 | 20 | 30 |
| L7 | 35 | 27.5 | 35 |
| L8 | 0 | 25 | 25 |
| HS | 10 | 10 | 10 |

The camera lens is responsible for focusing the HS focal plane on the Flea2 camera sensor. The design considerations are the same as for the Placido Corneal Topography camera optics design. The magnification is image an 8 mm pupil onto the 4.8×3.6 mm sensor. This gives a magnification m=0.4. Using an object lens of 100 mm, the second imaging lens is 40 mm focal length. The clinician or researcher can populate the MOMS with currently needed features and reserve the opportunity to expand capabilities as their needs change or as new correction modalities become available. Importantly, this strategy provides a mechanism for accelerating the transition of laboratory research into clinical practice by allowing clinicians to a) expand their instrument capabilities as research brings new treatments on-line and b) employ the same instrumentation used in the development of the new treatment.

The driving philosophy behind the proposed MOMS device is to meet the general needs of routine clinical practice, thereby increasing the general commercial viability, as well as the unmet needs of correcting the highly aberrated eye, and in particular the design of wavefront-guided corrections (e.g., soft lenses for the highly aberrated eye, refractive surgery, IOLs, inlays, onlays, etc.).

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An ocular measurement system for determining the front shape and power of the cornea of an eye comprising a Placidocorneal topography measurement system;
a projection corneal topography measurement system and an ocular wavefront measurement system.

2. The ocular measurement system of claim 1 wherein said Placido corneal topography system includes a device constructed and arranged for dynamic limbal detection, a device constructed and arranged for pupil detection and a device constructed and arranged for contact lens detection.

3. The ocular measurement system of claim 1 wherein said Placido corneal topography system includes a plurality of beam splitters, a camera and a first light source;
said plurality of beam splitters and said first camera are aligned with an axis of an object being measured; said first light source is offset with respect to said axis.

4. The ocular measurement system of claim 3 wherein said Placido corneal topography system includes a device constructed and arranged to introduce fluorescein into said object being measured, a second light source, said second light source offset with respect to said axis, a grating located between said second light source and said object being measured.

5. The ocular measurement system of claim 4 including a second camera, said second camera being offset with respect to said axis, said second camera receiving light rays from said second light source which have been reflected off said object being measured and deflected by one of said beam splitters.

6. The ocular measurement system of claim 1 wherein said ocular wavefront measurement system includes a third light source;
a beam coordinator;
a beam splitter;
a plurality of lens;
a beam rotator;
a Hartman screen wavefront sensor and a third camera.

7. The ocular measurement system of claim 6 wherein said third light source is a super luminescent diode.

8. A process for determining the front shape and power of the cornea of an eye comprising employing a Placidocornal topography measurement system;
employing a projection corneal topography measurement system and employing an ocular wavefront measurement system.

9. The process of claim 8 including dynamic limbal detection utilizing said Placido corneal topography system; pupil detection and contact lens detection.

10. The process of claim 8 wherein said limbal detection includes a plurality of beam splitters, a camera and a first light source; aligning said plurality of beam splitters and said first camera with an axis of said eye; offsetting said first light source with respect to said axis.

11. The process of claim 10 including introducing fluorescein into said eye, providing a second light source, offsetting said second light source with respect to said axis, providing a grating, positioning said grating between said second light source and said eye.

12. The process of claim 11 including proving a second camera, positioning said second camera offset with respect to said axis, said second camera receiving light rays from said second light source which have been reflected off said object being measured and deflected by one of said beam splitters.

13. The process of claim 8 including providing a third light source; providing a beam coordinator; providing a beam splitter; providing a plurality of lens; providing a beam rotator;
providing a Hartman screen wavefront sensor and providing a third camera.

14. The process of claim 13 including providing a super luminescent diode.
